(19) United States
(12) Patent Application Publication
(10) Pub. No.: US 2010/0318056 A1
(43) Pub. Date: Dec. 16, 2010

(54) COMBINATION PAD AND PANTY SHIELD WITH RAISED CHANNEL

(76) Inventors: Valerie Tucker, Cleveland, OH (US); Erica D. Burlin, Cleveland, OH (US)

Correspondence Address: HOWARD M COHN;PATENT ATTORNEY LLC, 21625 CHAGRIN BLVD #220, CLEVELAND, OH, 44122, US

(21) Appl. No.: 12/484,475

(22) Filed: Jun. 15, 2009

(51) Int. Cl. A61F 13/58

(52) U.S. Cl. 604/387

(57) ABSTRACT

A combination absorbent pad and panty shield with raised channel designed to be secured to an undergarment having a crotch region with a pair of side edges includes a liquid permeable cover; a liquid-impermeable cover; a liquid-impermeable baffle having a center section and opposite end sections that form a front end and a back end of the panty shield; an absorbent disposed in the central section of the baffle and enclosed by the cover to form an absorbent pad; first and second flaps mounted to opposite sides of the baffle and sized to depend downwardly and inwardly around the crotch portion of the undergarment; and a raised projection which projects upward from the liquid permeable cover and forms a ridge that extends along a centerline x-x that extends along the length of the combination pad and panty shield and substantially bisects the width of the combination pad and panty shield.

### TECHNICAL FIELD OF THE INVENTION

The present invention relates generally to an absorbent article such as a sanitary napkin, a panty liner, or an incontinent garment, and more particularly to a combination pad and panty shield with a with raised channel for full coverage protection.

### BACKGROUND OF THE INVENTION

Absorbent articles such as sanitary napkins, panty-liners, and incontinence pads are devices that are typically worn in the crotch region of an undergarment. These devices are designed to absorb and retain liquid and other discharges from the human body and to prevent body and clothing soiling. Sanitary napkins are a type of absorbent article worn by women in a pair of panties that are normally positioned between the wearer's legs, adjacent to the perineal area of the body. Sanitary napkins both with and without side flaps (or wings) are disclosed in the literature and are available in the marketplace.

Generally when sanitary napkins are provided with flaps, the flaps extend laterally from a central absorbent means and are intended to be folded around the edges of the wearer's panties in the crotch region. Thus, the flaps are disposed between the edges of the wearer's panties in the crotch region and the wearer's thighs. Commonly, the flaps are provided with an attachment means for either affixing the flaps to the underside of the wearer's panties or to the opposing flap. The flaps are particularly effective for preventing exudates from soiling the edges of the wearer's panties.

Functionally, sanitary napkins, catamenial pads and feminine pads are designed to absorb a greater quantity of body fluid and are made to he worn for longer periods of time than smaller external products such as panty-liners and panty shields.

In order to adequately perform their function, such absorbent articles should be positioned and retained close to the crotch region of the human body. The closer an absorbent article is held to or against the body, the better it is able to intercept discharged body fluid with less chance of leakage. It is therefore desirable to manufacture an absorbent article which contains a structure that will enable it to be securely held against the crotch region of the human body and to move with the body as the torso and thighs are manipulated and moved.

One of the main problems with monthly menstrual discharge is the embarrassing leaks or stained underwear that often occur. Due to the ineffective design of conventional sanitary pads, many women find themselves wearing panty liners in addition to their sanitary pad or wrapping a wadding tissue into their undergarments as a means of attempting to prevent leaks and stains from occurring. Not only is the use of a wadding tissue problematic, especially when the woman shifts positions, this extra padding can feel bulky and uncomfortable.

### ASPECTS AND SUMMARY OF THE INVENTION

It is an aspect of the present invention to provide combination pad and panty shield with a raised channel for full protection during a heavy menstrual cycle.

It is another aspect of the present invention to provide combination pad and panty shield with a raised channel that is comfortable to wear.

It is yet another aspect of the present invention to provide a combination pad and panty shield with a raised channel that attaches to an undergarment.

It is still another aspect of the present invention to provide a combination pad and panty shield with a raised channel that will conveniently eliminate any leaks of menstrual fluid that can occur when a woman shifts from one position to another after a lengthy period of time.

According to the present invention, there is disclosed a new design for a disposable sanitary pad and panty shield in one with a raised channel for the ultimate full coverage protection for any women that flows heavily during her menstruation or just want that extra protection during her monthly menstrual cycle. This design can be used for day wear or as an overnight pad. Because women come in all shapes and sizes, it will be available in several sizes such as large and small.

Further according to the present invention, there is disclosed a personal disposable sanitary device for women to protect their undergarment from menstrual discharge. The protective article includes a thin absorbent cotton sheet formed as a semi-thin pad which is very absorbent and will have its own panty shields that extend from the back and front of the main pad and which will adhere to the wearer's undergarment. It will have side shields that will wrap around the longitudinal edge of the crotch area of the undergarment, and a built in raised soft channel made in the center of the main pad that extends to the back panty shield that will fit like a thong to absorb trapped fluid as the wearer moves from the position from sitting to standing. The very top sheet of the article is covered with a dry weave material so that the new protective article will feel dry to the wearer. The bottom of the article can be lined with a protective plastic that will have adhesive to keep the article in place and from shifting from the wearer's undergarment during use. These features promote confidence in a pad protection throughout the day or night.

Further, according to the present invention, the combination pad and panty shield with a raised channel is designed with multiple layers. The bottom layer is constructed from a plastic material that features a conventional adhesive backing, which is used to secure the product to the undergarment and to provide a leak-proof barrier. Wrap around side tabs prevent the pad from shifting. These side tabs will also feature the adhesive. A thin cotton sheet is placed on top of the plastic backing, in the center of the unit, providing absorbency and adding structure to the rest of the design. Next, the raised cotton channel, constructed similar to a tampon and measuring approximately ¼″ in diameter, is placed to fit securely and comfortably within the crotch of the wearer. The raised channel will soak up menstrual fluid and act like a channel to direct the fluid onto the main pad and panty shields. It will extend to the rear of the unit and fit between the buttocks to absorb any escaped menstrual fluid. This rear area will be free to move from side to side to ensure a proper fit. The main pad is placed over the previous layers. It is approximately the same size and thickness of a conventional overnight sanitary pad. The top layer of the product is the traditional dry weave material that will keep the user feeling dry and clean. This covers the entire unit, creating a panty liner at the front and rear of the absorbent article with raised channel. While exact dimensions can be determined upon manufacture, the absorbent article with raised channel could be offered in two sizes, with the large size measuring a total of 14″ long and tapering from 8″ wide in the rear to 6″ wide at the front of the product. The smaller sized absorbent article with raised channel can be 8″ long and tapers from 5″ wide at the rear to 4″ wide at the front of the article.

According to the present invention, there is disclosed a combination absorbent pad and panty shield with raised channel designed to be secured to an undergarment having a crotch region with a pair of side edges. The combination pad and panty shield comprise a liquid permeable cover; a liquid-impermeable baffle having a center section and opposite end sections that form a front end and a back end of the panty shield; an absorbent disposed in the central section of the baffle and enclosed by the cover to form an absorbent pad; first and second flaps mounted to opposite sides of the baffle and sized to depend downwardly and inwardly around the crotch portion of the undergarment; and a raised projection which projects upward from the liquid permeable cover and forms a ridge that extends along a centerline X-X that extends along the length of the combination pad and panty shield and substantially bisects the width of the combination pad and panty shield.

Further according to the present invention, the raised projection is an elongated, cylindrical strip of material between the absorbent and the liquid permeable cover that extends from one end of the absorbent closest to the front end of the panty shield and towards the back end of the panty shield so that a length of the cylindrical strip of material extending out on the back end of the panty shield is not covered by the absorbent.

Still further according to the present invention, the cylindrical strip of material is an absorbent material.

Yet further according to the present invention, the cylindrical strip of material is disposed between the absorbent and a layer of absorbent material disposed against the inner facing surface of the baffle.

Also according to the present invention, the layer of absorbent material disposed against the inner facing surface of the baffle is enclosed between the absorbent and the baffle.

According to the present invention, the liquid permeable cover and the liquid-impermeable baffle form a raised oval shaped side extending around the perimeter of the liquid permeable cover.

Further according to the present invention, there are attachment means for securing the pair of flaps to the undergarment. The attachment means can be an adhesive located on both the first and second flaps. The attachment means can be a patch of adhesive located on the inner surface of the first flap and the outer surface of the second flap.

Still further according to the present invention, an adhesive to the outer facing surface of the baffle adapted to adhering to the wearer's body facing portion of their undergarment.

### BRIEF DESCRIPTION OF THE DRAWINGS

The structure, operation, and advantages of the present invention will become further apparent upon consideration of the following description taken in conjunction with the accompanying figures (Figures). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, often both reference numerals and legends (labels, text descriptions) will be used to identify elements. If legends are provided, they are intended merely as an aid to the reader, and should not in any way be interpreted as limiting.

FIG. 1 is a schematic side view of a human torso sliced vertically in half and shows an absorbent article retained adjacent to the crotch region by an undergarment.

FIG. 2 is a top view of the combination pad and panty shield with a raised channel, in accordance with the present invention.

FIG. 3 is a cross-sectional view of the combination pad and panty shield with a raised channel shown in FIG. 2 taken along line 3-3.

FIG. 4 is a top view of the barrier used in the construction of the combination pad and panty shield with a raised channel, in accordance with the present invention.

FIG. 5 is a top view of the barrier with layers of adhesive on an outer side used in the construction of the combination pad and panty shield with a raised channel, in accordance with the present invention.

FIG. 6 is a top view of the barrier used in the constriction of the combination pad and panty shield with a raised channel including a piece of absorbent material on the inner surface of the barrier, in accordance with the present invention.

FIG. 7 is a top view of the barrier used in the constriction of the combination pad and panty shield with a raised channel with a cylindrical strip of absorbent material disposed on the piece of absorbent material, in accordance with the present invention.

FIG. 8 is a top view of the barrier used in the construction of the combination pad and panty shield with a raised channel and a layer of absorbent material covering the cylindrical strip of absorbent material disposed on the layer of absorbent material, in accordance with the present invention.

FIG. 9 is a top view of the barrier used in the of the combination pad and panty shield with a raised channel, wherein the absorbent has been pressed into place to form a pattern including the raised channel extending the length of the absorbent and projecting outward there from, in accordance with the present invention.

FIG. 10 is a top view of the combination pad and panty shield with raised channel, showing the pressed pattern of the main pad and the raised channel wherein a sheet of dry weave material covers the outer surface of the article, in accordance with the present invention.

### DETAILED DESCRIPTION OF THE PREFERRED EMBODIMENTS

In the description that follows, numerous details are set forth in order to provide a thorough understanding of the present invention. It will he appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

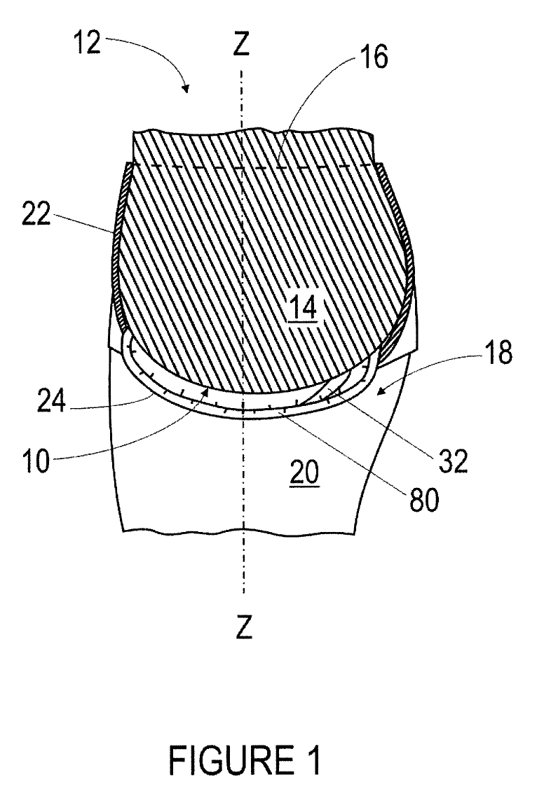

Referring to FIG. 1, a combination pad and panty shield with a raised channel 10 is shown positioned adjacent to the crotch region of a human body 12. The combination pad and panty shield with a raised channel is an absorbent article that can include a sanitary napkin, a catamenial pad, a feminine pad, a pantyliner, a panty shield, an incontinent garment, or some other type of absorbent product. For purposes of discussion, the combination pad and panty shield with a raised channel 10 will be described as a sanitary napkin and shield. The sanitary napkin and shield 10 is designed to be worn by women to absorb discharged body fluid, especially menstrual fluid and urine.

In FIG. 1, a side view of a human body 12 is shown which includes a torso 14 having a waist 16 and a crotch region 18 located at a lower end of the torso. A pair of legs 20 (one of which is shown) depend downward from the crotch region 18. The sanitary napkin and shield 10 is retained in close proximity to the pudendum or vulva area of a woman by an undergarment 22. The undergarment 22 is a typical pair of underpants having a waist opening, two leg openings and a crotch portion located between the two leg openings. The crotch portion has edges or seams 24 which are located on opposite sides of the crotch portion. The edges or seams 24 extend around each of the leg openings. A portion of the edges or seams 24 normally contact the groin of the wearer when the undergarment 22 is pulled up around the torso 14 and adjusted about the waist 16. The forces acting on the edges or seams 24 of the undergarment 22 in the crotch region 18 cause an upward force which holds the edges or seams 24 in contact with the wearer's body.

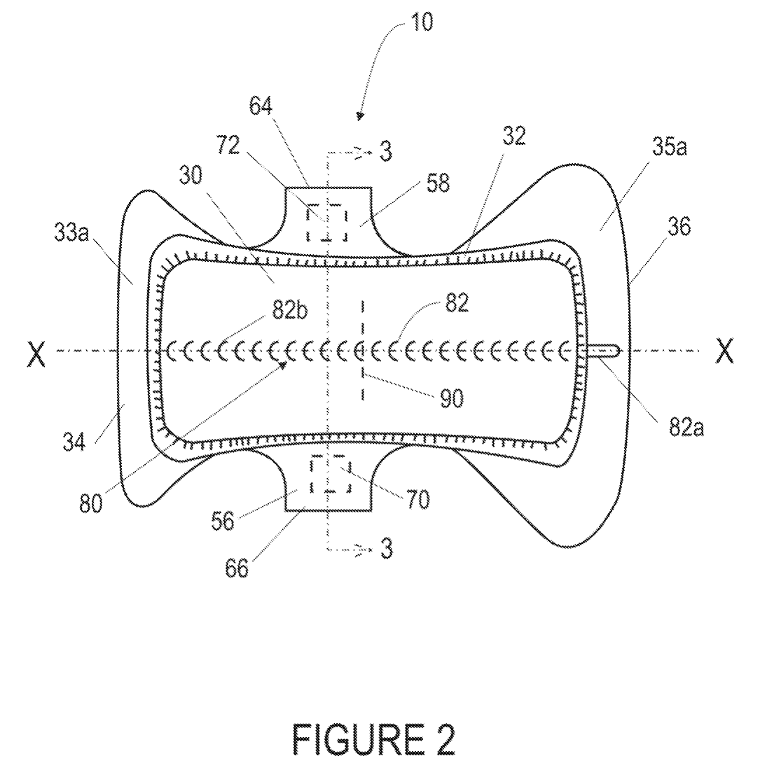

Referring to FIG. 2, the sanitary napkin and shield 10 is shown having a body contacting surface 30 with a raised generally rectangular extending side 32. The sanitary napkin and shield 10 also has a first end 34 and a second end 36. The portions of the 33 and 35 which extend from the side 32 to the outer periphery of the sanitary napkin and shield 10, 34 and 36, respectively, form the panty shield.

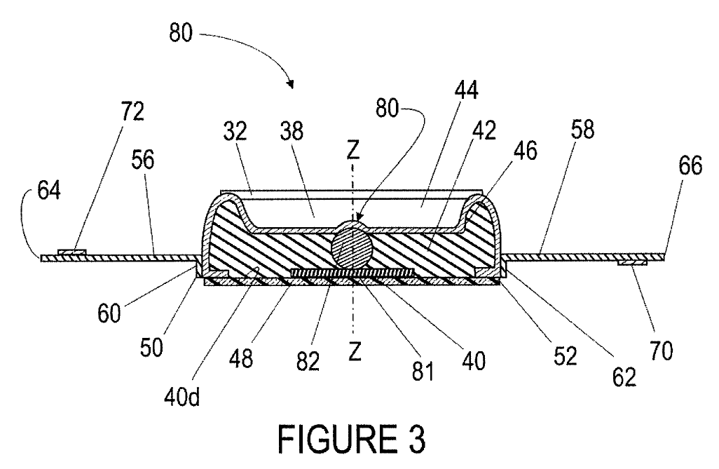

As best seen in FIG. 3, the combination pad and panty shield with a raised channel 10 includes a liquid permeable cover 38 and a liquid-impermeable baffle 40 which cooperate to enclose an absorbent pad 42. The cover 38, the baffle 40 and the absorbent pad 42 are formed into the sanitary napkin and shield 10. The raised, generally rectangular extending side 32 is formed on the upper or body contacting surface 44. The raised generally oval extending side 32 has a vertically oriented outer surface 46. “Vertically oriented” means that the outer surface 46 can be aligned parallel to the central vertical axis z-z or be aligned at an acute angle thereto. It should be noted that the vertical outer surface 46 can be linear or non-linear. When non-linear, the outer surface 46 can be curved or arcuate in configuration, as shown in FIG. 2.

The combination pad and panty shield with a raised channel pad 10 also has a garment facing surface 48 aligned opposite to the body facing surface 44. The garment facing surface 48 is designed to face the inner crotch portion of an undergarment.

In FIG. 3, one will notice that the cover 38 and the baffle 40 are joined together at locations 50 and 52 which are located on the garment facing surface 48. However, they can also be adjoined along the vertical outer surface 46 if desired. Alternatively, the baffle 40 and the absorbent 42 can be completely enclosed by the cover 38 if desired.

The combination pad and panty shield with a raised channel pad 10 also contains a pair of flaps 56 and 58 which have sufficient size to extend around at least a portion of the crotch of an undergarment. Preferably, the flaps 56 and 58 will overlap and be attached to one another. Alternatively, the flaps 56 and 58 can be attached or secured to the outer surface of the crotch portion of an undergarment. Each of the flaps, 56 and 58, respectively, has a proximal edge, 60 and 62, respectively, and a distal edge, 64 and 66, respectively. Each of the proximal edges 60 and 62 are affixed to the vertically oriented outer surface 46. Each of the distal edges 64 and 66 extend downward and inward around the crotch portion of an undergarment. It is important to note that the flaps 56 and 58 are not designed to extend upward into the groins of the user since the raised longitudinally-extending sides 46 will serve that function. Instead the pair of flaps 56 and 58 depend downwardly and inwardly around the crotch portion of an adjacent undergarment so as to prevent staining of the undergarment by any body fluid that may leak off of the absorbent article 10. The pair of flaps 56 and 58 can be made into any desired size and shape. Preferably, provided that they have a width along the transverse central axis Y-Y sufficient to overlap when they wrap around the undergarment. For best results, the flaps 56 and 58 should be centrally located on the combination pad and panty shield with a raised channel pad 10 such that they bifurcate the central transverse axis Y-Y.

Referring again to FIGS. 2 and 3, each of the flaps 56 and 58 includes an attachment means 70 and 72 for securing the pair of flaps 56 and 58 to one another. In FIGS. 2 and 3, the attachment means 70 is shown to be a patch of adhesive located on the outer surface of the flap 56 while the attachment means 72 is a patch of adhesive located on the inner surface of the flap 58. The flap 56 is designed to be folded around the undergarment first and then the second flap 58 is designed to be folded around the undergarment such that the adhesive 70 can contact adhesive 72 and the pair of flaps 56 and 58 can be held securely about the undergarment. It is also possible to make the flaps 56 end 58 shorter so that they do not overlap but instead have an attachment means 70 and 72 which are designed to be attached directly to the outer surface of the undergarment. When the flaps 70 and 72 are to be attached to the undergarment, the attachment means 70 and 72 are located on the inner surface of each flap 56 and 58. Besides the use of adhesives, the attachment means 56 and 58 can be a mechanical fastener such as a hook and loop type fastener. VELCRO™. is one type of hook and loop fastener. “VELCRO” is a registered trademark of Velcro USA Inc. Other forms of fasteners include materials which will adhere to one another. For example, one could form the entire flap or a portion of each of the flaps, 56 and 58 respectively, out of a material that will adhere to another material. A cohesive-adhesive would cause the two flaps 56 and 58 to adhere to one another yet still be releasable.

Referring to FIGS. 2 and 3, there is illustrated a raised projection 80 which projects upward from the liquid permeable cover 38 and forms a ridge that extends along the centerline x-x that extends along the length of the combination pad and panty shield with a raised channel 10 and substantially bisects the width of the combination pad and panty shield. The raised projection 80 is formed by a flat rectangular shaped layer of material 81 disposed against the inner facing surface 40d of the battle 40. The layer of material 81 is constructed of an absorbent material, such as cotton. Raised projection 80 also includes an elongated, cylindrical strip 82 of material, having a construction similar to a tampon that extends from the rectangular extending side 32 closest to the front end 34 of the panty shield and towards the back end 36 of the panty shield and beyond the raised, generally rectangular extending side 32 toward the second end 36.

The cylindrical strip 82 will be of an absorbent material, such as cotton, to form a soft and absorbent layer that will draw in menstrual fluid in a manner similar to a tampon and it will act like a channel to direct the menstrual fluid onto the main pad 42. The flow of the menstrual fluid is caused by the female anatomy when the raised channel 80, along with the main pad 30 is fitted softly and comfortably adjacent to the crotch of the wearer. The rear portion 82a of the raised cylindrical strip 82 extends in the back panty shield 35 and will fit softly and comfortably between the wearer's buttocks to absorb escaped menstrual fluid and act like a channel to draw the menstrual fluid back into the main pad 30.

In the embodiment of the combination pad and panty shield with a raised channel 10, as shown in FIGS. 2 and 3, the rear portion 82a of raised channel 80 will be free to move about from side to side on the back panty shield 35 for a sure fit. The raised channel 82b formed along the center line X-X of the article 10 within the perimeter of the rectangular extending side 32, will channel the menstrual fluid into the main section 30 of the panty shield to keep the panty shield clean and dry.

It should be noted that during use, the combination pad and panty shield with a raised channel 10 can be centered along the vertical central axis z-z of the body, as is shown in FIG. 1, or it can be positioned forward or rearward of the vertical central axis of the body, depending upon the wearers particular preference. It should also be noted that although the sanitary napkin 10 is depicted as being symmetrical in shape with the first and second ends, 34 and 36 respectively, being equally spaced from the center point 90, it is possible to form a non-symmetrical sanitary napkin which varies in shape from one end to the other.

To better appreciate the construction of the improved, panty shield 10, an exemplary step by step, description follows.

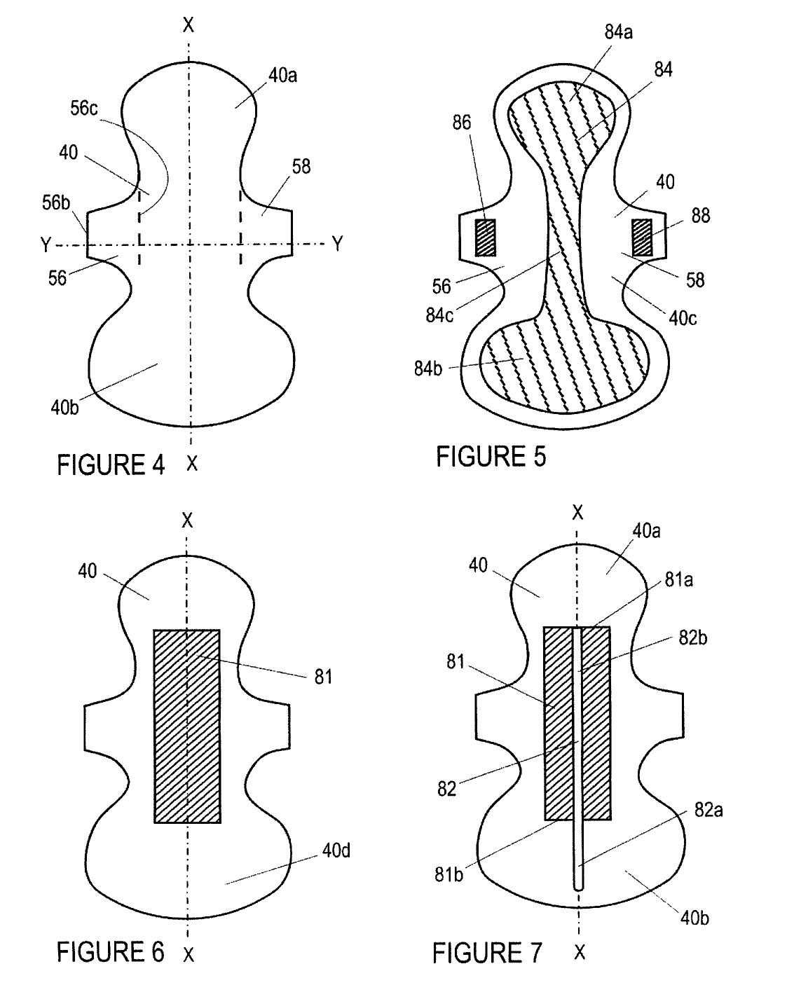

Referring to FIG. 4, the sanitary napkin 10 includes a liquid-impermeable baffle 40, such as a sheet of plastic, to provide a leak proof barrier to the wearer's undergarment from menstrual fluid and is used to support the remainder of the napkin 10. The length for the baffle 40 can be between about 12 inches to about 16 inches. The front end 40a of baffle 40 can be about 6 inches to about 8 inches in width. The back end 40b of the baffle 40 can be about 8 inches to about 12 inches in width. The side shields 56 and 58 are about 2 inches wide and about 3½ inches in length (the distance from where the shield 56 intersects the main baffle 56a to the outer edge 56b).

Referring to FIG. 5, there is illustrated the placement of one or more layers 84, 86 and 88 of an adhesive to the outer facing surface 40c of the baffle 40. The adhesive layers 84,86 and 88 allows the absorbent article 10 to contour to and adhere to the wearer's body facing portion of their undergarment. The adhesive for the layers 84,86 and 88 is selected so that the absorbent article 10 can be attached to and then taken off the user's under garment. The width of the adhesive layers 84a and 84b will have to be wide enough on the front of the panty shield and on the back of the panty shield to stay in place and to keep the shields 33 and 35 unnoticeable. The adhesive layer 84c will be about 1 inch or 1¼ inches in width so that tile article 10 will adhere to the crotch of the user's underwear. For the side shields 56 and 58, the layers 86 and 88 of adhesive allow the side shields to fold down and over the undersides of the wearer's undergarment and then adhered to each other or the undergarment so as to keep article 10 in place. This placement ensures that the longitudinal side edges of the undergarment are kept clean and dry from leaks and stains. The adhesive layers 84,86 and 88 will both keep the article 10 in place on the user's underwear and also keep the article 10 from bunching up. This ensures that the front and back panty shields remained unnoticeable.

In FIG. 6, a layer 81 of absorbent material, such as cotton, is placed on the inner facing surface 40d of the baffle 40 along the axis X-X and is adapted to be located opposite the wearer's crotch. The layer 81 is very absorbent and will help keep the shape of the absorbent pad 42 during the wearing of the article 10, which will make the finished product more comfortable to the wearer.

In FIG. 7, the cylindrical strip 82 that forms raised channel 80 is placed along the axis X-X on the center of the outer facing service 81a of the layer 81, so as to be located facing towards the user's body. The raised channel 80 includes the section 80 will extend from the front end 81a of the absorbent layer 81, beyond the rear end 81b, and into the end of section 40b of baffle 40 of the article 10.

The cylindrical strip 82 can have a length of about 12 inches to about 14 inches and a diameter of about ½ to about 1 inch. The strip 82 will form a soft and absorbent layer that will draw in menstrual fluid, in a manner similar to a tampon, and will act as a channel to direct the fluid onto the main pad 42. This action is done by the female anatomy when the raised cotton channel 80, along with the main pad 42, are fitted softly and comfortably in the crotch area of the wearer. The raised channel 82a, in the back of the article 10, will fit softy and comfortably between the wearer's buttocks to absorb escaped menstrual fluid and act like a channel to draw the menstrual fluid into the absorbent 42. In the finished product, the raised channel 82a will be free to move about from side to side for a sure fit. The raised channel 82b above the main pad 42 will channel the menstrual fluid into the absorbent 42 to keep the user's undergarment clean and dry.

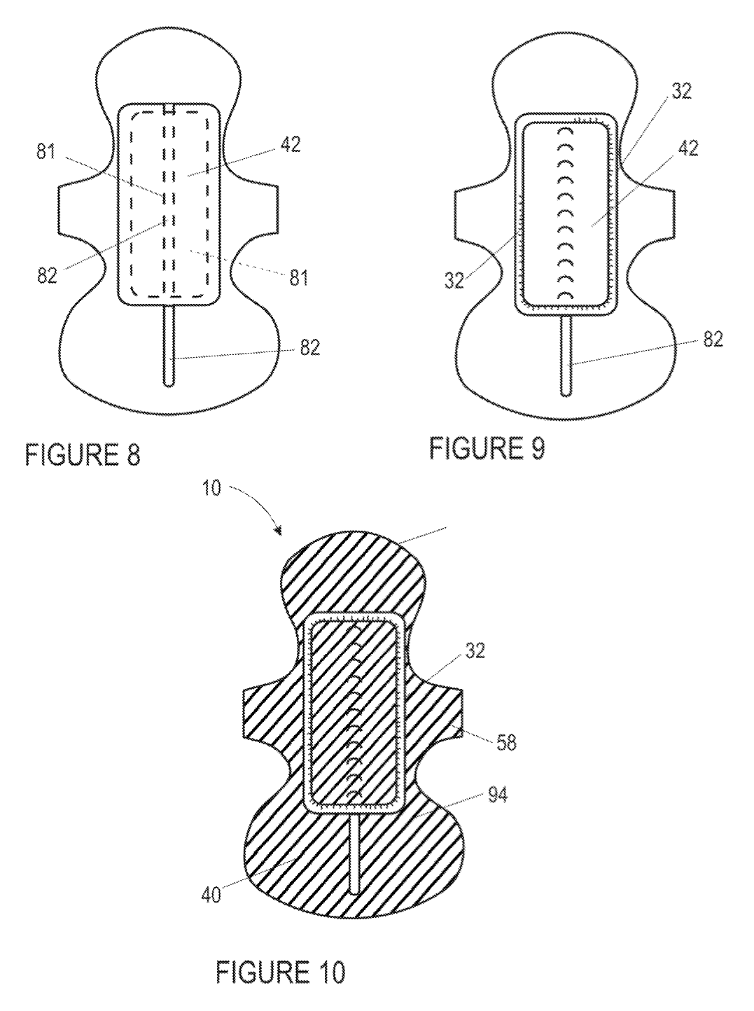

In FIG. 8, the main pad 42 is placed over the layer 81 and the raised channel strip 82. The pad 42 can be about 12 inches in length. The front end and back end of pad 42 can be about be 3½ inches wide. For the most superior comfort, the raised channel will be cushioned by the semi-thin pad 42 that is very absorbent.

In FIG. 9, the pad 42 and the strip 82 are pressed onto the baffle 40 whereby the perimeter of the pad can font an upstanding ridge 32 and the ridge 82. The pressed indention within the borders of the ridge 32 will act as a guard against leaks and it will also act as comfort cushions between the crotch of the wearer.

In FIG. 10, a layer 94 of absorbent material having a dry weave pattern is placed over the inward facing side of baffle 40 to provide the completed article 10. The layer 94 will keep the user feeling dry and clean from wetness. Because the outer facing sides of article 10 are constructed of the plastic baffle 40 and the layer 94 of dry weave material, together they are not seen when the article is disposed on an undergarment. This gives the wearer confidence to move throughout their day feeling fresh and dry.

The dry weave material on the side shields 56 and 58, will help keep the longitudinal edge of tile wearer's panties clean and dry. In the completed article, the front and back panty shields 33 and 35 are very noticeable. The front panty shield 33 extends from the upstanding ridge 32 to the front edge 34 of the article 10. The back panty shield 35 extends from the upstanding ridge 32 to the rear edge 36 of the article 10. The front and back panty shields 33 and 35 can be secured and removed from the wearer's front and back part of their underwear by the adhesive 84.

The front panty shield 33 is about 6 inches wide and about 1 inch in length from the main pad 40 to the front edge 34. The back panty shield 35 is about 8 inches across and 2½ inches in length from the main pad 40 to the rear edge 36.

Regarding the use of combination pad and panty shield with raised channel 10, its unique shape will remove any confusion as to how to secure the article to the user's underwear. The smallest end 33 is placed on the front on the user's undergarment, the larger end 35 is pressed on the back part of the wearer's undergarment. Then, the side shields 56 and 58 are folded under the longitudinal edge of the user's underwear so that the absorbent 40 is appropriately placed for full coverage protection.

It is also understood, of course, that while the form of the invention herein shown and described constitutes a preferred embodiment of the invention, it is not intended to illustrate all possible forms thereof. It will also be understood that the words used are words of description rather than limitation, and that various changes maybe made without departing from the spirit and scope of the invention disclosed. Thus, the scope of the invention should be determined by the appended claims and their legal equivalents, rather than solely by the examples given.

1. A combination absorbent pad and panty shield with raised channel designed to be secured to an undergarment having a crotch region with a pair of side edges, comprising:
a) a liquid permeable cover;
b) a liquid-impermeable baffle having a center section and opposite end sections that form a front end and a back end of the panty shield;
c) an absorbent disposed in the central section of the baffle and enclosed by the cover to form an absorbent pad;
d) first and second flaps mounted to opposite sides of the baffle and sized to depend downwardly and inwardly around the crotch portion of the undergarment; and
e) a raised projection which projects upward from the liquid permeable cover and forms a ridge that extends along a centerline X-X that extends along the length of the combination pad and panty shield and substantially bisects the width of the combination pad and panty shield.

2. The combination absorbent pad and panty shield with raised channel of claim 1 wherein the raised projection is an elongated, cylindrical strip of material between the absorbent and the liquid permeable cover that extends from one end of the absorbent closest to the front end of the panty shield and towards the back end of the panty shield so that a length of the cylindrical strip of material extending out on the back end of the panty shield is not covered by the absorbent.

3. The combination absorbent pad and panty shield with raised channel of claim 2 wherein the cylindrical strip of material is an absorbent material.

4. The combination absorbent pad and panty shield with raised channel of claim 2 wherein the cylindrical strip of material is disposed between the absorbent and a layer of absorbent material disposed against the inner facing surface of the battle.

5. The combination absorbent pad and panty shield with raised channel of claim 4 wherein the layer of absorbent material disposed against the inner facing surface of the baffle is enclosed between the absorbent and the baffle.

6. The combination absorbent pad and panty shield with raised channel of claim 5 wherein the liquid permeable cover and the liquid-impermeable baffle form a raised oval shaped side extending around the perimeter of the liquid permeable cover.

7. The combination absorbent pad and panty shield with raised channel of claim 1 further including attachment means for securing the pair of flaps to the undergarment.

8. The combination absorbent pad and panty shield with raised channel of claim 7 wherein the attachment means is an adhesive located on both the first and second flaps.

9. The combination absorbent pad and panty shield with raised channel of claim 8 wherein the attachment means is a patch of adhesive located on the inner surface of the first flap and the outer surface of the second flap.

10. The combination absorbent pad and panty shield with raised channel of claim 1 wherein an adhesive to the outer facing surface of the baffle adapted to adhering to the wearer's body facing portion of their undergarment.

11. A combination absorbent pad and panty shield with raised channel designed to be secured to an undergarment having a crotch region with a pair of side edges, comprising:
a) liquid permeable cover;
b) a liquid-impermeable baffle having a center section and opposite end sections that form a front end and a back end of the panty shield;
c) an absorbent disposed in the central section of the baffle and enclosed by the cover to form an absorbent pad;
d) first and second flaps mounted to opposite sides of the baffle and sized to depend downwardly and inwardly around the crotch portion of the undergarment;
e) a raised oval shaped side extending around the perimeter of the liquid permeable cover; and
f) a raised projection which projects upward from the liquid permeable cover and forms a ridge that extends along a centerline x-x that extends along the length of the combination pad and panty shield and substantially bisects the width of the combination pad and panty shield.

12. The combination absorbent pad and panty shield with raised channel of claim 11 wherein the raised projection is an elongated, cylindrical strip of material between the absorbent and the liquid permeable cover that extends from one end of the absorbent closest to the front end of the panty shield and towards the back end of the panty shield so that a length of the cylindrical strip of material extending out on the back end of the panty shield is not covered by the absorbent.

13. The combination absorbent pad and panty shield with raised channel of claim 12 wherein the cylindrical strip of material is an absorbent material.

14. The combination absorbent pad and panty shield with raised channel of claim 12 wherein the cylindrical strip of material is disposed between the absorbent and a layer of absorbent material disposed against the inner facing surface of the baffle.

15. The combination absorbent pad and panty shield with raised channel of claim 14 wherein the layer of absorbent material disposed against the inner facing surface of the baffle is enclosed between the absorbent and the baffle.

16. The combination absorbent pad and panty shield with raised channel of claim 15 wherein the raised oval shaped side extending around the perimeter of the liquid permeable cover liquid is formed of the permeable cover and the liquid-impermeable baffle.

17. The combination absorbent pad and panty shield with raised channel of claim 11 further including attachment means for securing the pair of flaps to the undergarment.

18. The combination absorbent pad and panty shield with raised channel of claim 17 wherein the attachment means is an adhesive located on both the first and second flaps.

19. The combination absorbent pad and panty shield with raised channel of claim 18 wherein the attachment means is a patch of adhesive located on the inner surface of the first flap and the outer surface of the second flap.

20. The combination absorbent pad and panty shield with raised channel of claim 11 wherein an adhesive to the outer facing surface of the baffle adapted to adhering to the wearer's body facing portion of their undergarment.
